(19) United States
(12) Patent Application Publication
(10) Pub. No.: US 2024/0115206 A1
(43) Pub. Date: Apr. 11, 2024

(54) ALTERNATIVE PLACEMENT OPTIONS FOR SMART IMPLANTS

(71) Applicant: Warsaw Orthopedic, Inc., Warsaw, IN (US)

(72) Inventors: Steven D. Glassman, M.D., Louisville, KY (US); Newton H. Metcalf, JR., Memphis, TN (US); Jerald L. Redmond, Germantown, TN (US); Arjun Siby-Kurian, Memphis, TN (US); Adam D. Glaser, Colliersville, TN (US)

(73) Assignee: Warsaw Orthopedic, Inc., Warsaw, IN (US)

(21) Appl. No.: 18/542,215

(22) Filed: Dec. 15, 2023

Related U.S. Application Data
(63) Continuation-in-part of application No. 18/183,484, filed on Mar. 14, 2023.
Application No. 18/183,484 is a continuation-in-part of application No. 18/068,140, filed on Dec. 19, 2022.
Application No. 18/068,140 is a continuation-in-part of application No. 18/062,867, filed on Dec. 7, 2022.
(60) Provisional application No. 63/329,982, filed on Apr. 12, 2022.

(51) Int. Cl. A61B 5/00; A61B 5/01; A61B 5/07; A61B 17/70

(52) U.S. Cl. CPC A61B 5/686; A61B 5/01; A61B 5/076; A61B 5/4851; A61B 17/7032; A61B 2017/00734; A61B 2560/045; A61B 2562/028

(57) ABSTRACT

A spinal implant includes an attachment portion having an opening configured to capture a longitudinal member therein, wherein the attachment portion is the only attachment means of the spinal implant. The spinal implant further includes a housing integrally connected to the attachment portion and defining a sealed cavity for supporting a microelectronics assembly and a power source therein. The spinal implant further includes at least one antenna in electrical communication with the microelectronics assembly and at least one sensor in electrical communication with the microelectronics assembly, wherein the microelectronics assembly is configured to transmit information received from the at least one sensor to an external device using the at least one antenna.

### CROSS-REFERENCE TO RELATED APPLICATIONS

This application is a continuation-in-part of U.S. application Ser. No. 18/183,484, titled SPINAL IMPLANTS WITH ELECTRONICS CARTRIDGE AND EXTERNALIZED ANTENNA, filed Mar. 14, 2023, which claims priority to U.S. Provisional Application 63/329,982, titled SMART IMPLANT DESIGNS FOR HOUSING A POWER SOURCE, ANTENNA, GAUGES, AND MICROELECTRONICS, and filed Apr. 12, 2022. U.S. application Ser. No. 18/183,484 is also a continuation-in-part of U.S. application Ser. No. 18/062,867, titled SPINAL ROD CONNECTING COMPONENTS WITH ACTIVE SENSING CAPABILITIES, filed Dec. 7, 2022 and U.S. application Ser. No. 18/068,140, titled SPINAL IMPLANTS WITH ACTIVE SENSING CAPABILITIES, filed Dec. 19, 2022. The disclosures of these applications are incorporated herein by reference in their entirety.

### FIELD

The present disclosure generally relates to mechanical and electrical sensor assemblies and antenna designs for implant devices, and more particularly to application of sensor assemblies in locations of a spinal construct other than the head of a pedicle screw.

### BACKGROUND

Treatment of spinal disorders, such as degenerative disc disease, disc herniations, scoliosis or other curvature abnormalities, and fractures, often requires surgical treatments. For example, spinal fusion may be used to limit motion between vertebral members. As another example, implants may be used to preserve motion between vertebral members.

Surgical treatment typically involves the use of implants and longitudinal members, such as spinal rods. Implants may be disposed between two vertebral members for supporting and/or repositioning the vertebral members. Implants may also be used to facilitate a fusion process between a superior vertebra and an inferior vertebra. Longitudinal members may be attached to the exterior of two or more vertebral members to assist with the treatment of a spinal disorder. Longitudinal members may provide a stable, rigid column that helps bones to fuse, and may redirect stresses over a wider area away from a damaged or defective region. Also, rigid longitudinal members may help in spinal alignment.

Screw assemblies may be used to connect a longitudinal member to a vertebral member. A screw assembly may include a pedicle screw, hook, tulip bulb connector or other type of receiver, and a set screw, among other components. A pedicle screw can be placed in, above and/or below vertebral members that were fused, and a longitudinal member can be used to connect the pedicle screws which inhibit or control movement. A set screw can be used to secure the connection of a longitudinal member and a pedicle screw, hook, or other connector. Implants may include one or more sensors for monitoring post-operative aspects of the treatment and transmitting sensor data to an external reader. However, it would be advantageous to apply sensor systems to locations other than the head of pedicle screws, e.g., to sense load, temperature, or other aspects at a variety of locations throughout the spinal construct.

### SUMMARY

The techniques of this disclosure generally relate to sensor assemblies configured for alternative placement options and having various sensors for communicating sensed attributes to an external reader. In an example embodiment a spinal implant is disclosed. The spinal implant includes an attachment portion having an opening configured to capture a longitudinal member therein, wherein the attachment portion is the only attachment means of the spinal implant. The spinal implant further includes a housing integrally connected to the attachment portion and defining a sealed cavity for supporting a microelectronics assembly and a power source therein. The spinal implant further includes at least one antenna in electrical communication with the microelectronics assembly and at least one sensor in electrical communication with the microelectronics assembly, wherein the microelectronics assembly is configured to transmit information received from the at least one sensor to an external device using the at least one antenna.

Implementations of the disclosure may include one or more of the following optional features. In some examples, the spinal implant is configured to extend away from the longitudinal member in an axial direction when attached to the longitudinal member. The attachment portion may include a hook. The at least one antenna may be disposed within the housing. The at least one antenna may include a first antenna portion and a second antenna portion disposed on opposite sides of the housing. The power source may be a battery. The battery may be configured to be inductively recharged. In some examples, the housing includes a hermetically sealed, removable electronics cartridge having at least one antenna interface. The removable electronics cartridge may have a threaded exterior that mates with the housing. The at least one sensor may be disposed within the removable electronics cartridge.

In some examples, the at least one antenna is configured to flexibly extend away from the spinal implant. The at least one sensor may include at least one temperature sensor configured to measure a temperature of a surgical site. The at least one sensor may include at least one strain gauge configured to measure a localized force sensed by the spinal implant. The at least one sensor may be chosen from the group including: accelerometer, gyroscope, strain gauge, pressure sensor, pH sensor, impedance sensor, optical sensor, and temperature sensor. The opening may be a threaded U-shaped cavity. In some examples, the attachment portion further includes a threaded portion configured to receive a screw, thereby securing the spinal implant to the longitudinal member.

A surgical site monitoring system is disclosed, the surgical site monitoring system includes the example spinal implant embodiment disclosed above. The surgical site monitoring system further includes an external reader device configured to receive the information transmitted from the spinal implant. Implementations of the disclosure may include one or more of the following optional features. In some examples, the external reader device is configured to display at least a portion of the information transmitted from the spinal implant. The external reader device may be configured to provide a notification when one or more measurements transmitted from the spinal implant are outside of a value range. The external reader device may be configured to receive the information through an intermediate relay device.

### BRIEF DESCRIPTION OF DRAWINGS

FIG. 1 shows an example of alternative placement options.

FIGS. 2A-2C illustrate example implant components.

FIG. 3 illustrates an example digital pedicle screw system.

FIG. 4 illustrates an example sensor assembly.

FIG. 5 shows an exploded view of an example sensor assembly.

FIG. 6 shows an exploded view of an example sensor assembly.

FIG. 7 is a cross-section drawing of an example sensor assembly.

FIG. 8 illustrates an example of a surgical site monitoring system.

### DETAILED DESCRIPTION

Embodiments of the present disclosure relate generally, for example, to sensor systems configured for alternative placement options, i.e., sensors systems whose primary function is to provide telemetry rather than fixation. Embodiments of the devices and methods are described below with reference to the Figures.

The following discussion omits or only briefly describes certain components, features and functionality related to medical implants, installation tools, and associated surgical techniques, which are apparent to those of ordinary skill in the art. It is noted that various embodiments are described in detail with reference to the drawings, in which like reference numerals represent like parts and assemblies throughout the several views, where possible. Reference to various embodiments does not limit the scope of the claims appended hereto because the embodiments are examples of the inventive concepts described herein. Additionally, any example(s) set forth in this specification are intended to be non-limiting and set forth some of the many possible embodiments applicable to the appended claims. Further, particular features described herein can be used in combination with other described features in each of the various possible combinations and permutations unless the context or other statements clearly indicate otherwise.

Terms such as “same,” “equal,” “planar,” “coplanar,” “parallel,” “perpendicular,” etc. as used herein are intended to encompass a meaning of exactly the same while also including variations that may occur, for example, due to manufacturing processes. The term “substantially” may be used herein to emphasize this meaning, particularly when the described embodiment has the same or nearly the same functionality or characteristic, unless the context or other statements clearly indicate otherwise. The term “about” may encompass a meaning of being +/−10% of the stated value.

Unless otherwise specifically defined herein, all terms are to be given their broadest possible interpretation including meanings implied from the specification as well as meanings understood by those skilled in the art and/or as defined in dictionaries, treatises, etc. It must also be noted that, as used in the specification and the appended claims, the singular forms “a,” “an” and “the” include plural referents unless otherwise specified, and that the terms “comprises” and/or “comprising,” when used in this specification, specify the presence of stated features, elements, and/or components, but do not preclude the presence or addition of one or more other features, steps, operations, elements, components, and/or groups thereof.

The disclosed smart implants embodiments include sensor assemblies that are configured to provide telemetry to an external device. Telemetry data may include position/motion information, force/strain information, temperature, tissue impedance, and so forth. For example, spinal implants may be installed during a surgical procedure such as a spinal fusion and may be configured to sense post-operative aspects of the surgical procedure, such as forces between spinal implants or components. The spinal implants may provide telemetry related to the surgical site, such as temperature readings from a variety of locations around the surgical site. The spinal implants may provide telemetry related to position and/or motion of the patient's spine during surgery, or other indicators of fusion status. In these cases and others, an external reader, such as the system disclosed in U.S. patent application Ser. No. 16/855,444, incorporated herein by reference in its entirety, may display or otherwise provide the telemetry to, e.g., a medical professional for evaluation. In some examples, the reader is configured to be disposed at or near a patient's bedside, allowing the patient and the medical professional to readily observe the displayed status information. The external reader may also receive and may display telemetry from other sources such as, but not limited to, one or more wearable sensor system that are affixed to the patient. The reader device itself may include additional sensors as well.

The smart implants may include electronics, such as sensors or sensor systems which acquire the telemetry data, and transmitter (or transceiver) systems which transmit the telemetry to an external reader/receiver device. The smart implants may also include a power source, such as a battery (rechargeable or otherwise) for powering the electronics. The transmitter system may include an antenna for radiating the telemetry signal to the reader device (and/or an intermediate relay device). In some cases, the tissue surrounding the implant may constrain antenna configuration. For example, a rigid antenna extending away from a spinal implant may irritate or damage surrounding tissue. The level of irritation or damage may be related to the type of tissue as well as the construction and/or configuration of the antenna.

Referring to the disclosed embodiments generally, components of the implant systems can be fabricated from biologically acceptable materials suitable for medical applications, including metals, synthetic polymers, ceramics and bone material and/or their composites. The components may be fabricated using bio-inert materials, such as metals, ceramics, polymers, etc. The components may also be fabricated (either entirely or at least partially) using bio-resorbable or bio-convertible materials, as appropriate. For example, the components, individually or collectively, can be fabricated from materials such as stainless steel alloys, commercially pure titanium, titanium alloys, Grade 5 titanium, super-elastic titanium alloys, cobalt-chrome alloys, super-elastic metallic alloys (e.g., Nitinol, super-elastoplastic metals, such as GUM METAL®), ceramics and composites thereof such as calcium phosphate (e.g., SKELITE™), alumina, yttria-stabilized zirconia (YSZ), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyketide, polyglycolide, polytyrosine carbonate, polycaprolactone, polylactic acid or polylactide and their combinations.

Various components of the implant system may be formed or constructed with material composites, including the above materials, to achieve various desired characteristics such as strength, rigidity, elasticity, compliance, biomechanical performance, durability and radiolucency or imaging preference. The components of the present implant system, individually or collectively, may also be fabricated from a heterogeneous material such as a combination of two or more of the above-described materials. The components of the implant system may be monolithically formed, integrally connected or include fastening elements and/or instruments, as described herein. The components of the implant system may be formed using a variety of subtractive and additive manufacturing techniques, including, but not limited to machining, milling, extruding, molding, 3D-printing, sintering, coating, vapor deposition, and laser/beam melting.

Furthermore, various components of the implant system may be coated or treated with a variety of additives or coatings to improve biocompatibility, bone growth promotion or other features. Various embodiments and components may be coated with a ceramic, titanium, and/or other biocompatible material to provide surface texturing at (a) the macro scale, (b) the micro scale, and/or (c) the nano scale, for example. Similarly, components may undergo a subtractive manufacturing process such as, for example, grit blasting and acid etching, providing for surface texturing configured to facilitate osseointegration and cellular attachment and osteoblast maturation. Example surface texturing of additive and subtractive manufacturing processes may include (a) macro-scale structural features having a maximum peak-to-valley height of about 40 microns to about 500 microns, (b) micro-scale structural features having a maximum peak-to-valley height of about 2 microns to about 40 microns, and/or (c) nano-scale structural features having a maximum peak-to-valley height of about 0.05 microns to about 5 microns. In various embodiments, the three types of structural features may be overlapping with one another. Additionally, such surface texturing may be applied to any surface, e.g., both external exposed facing surfaces of components and internal non exposed surfaces of components. Further discussion regarding relevant surface texturing and coatings is described in, for example, U.S. Pat. No. 11,096,796, titled Interbody spinal implant having a roughened surface topography on one or more internal surfaces, and filed on Mar. 4, 2013—the entire disclosure of which is incorporated herein by reference in its entirety. Accordingly, it shall be understood that any of the described coating and texturing processes of U.S. Pat. No. 11,096,796, may be applied to any component of the various embodiments disclosed herein, e.g., the exposed surfaces and internal surfaces. Another example technique for manufacturing an orthopedic implant having surfaces with osteoinducting roughness features including micro-scale structures and nano-scale structures is disclosed in U.S. Pat. No. 10,821,000, the entire contents of which are incorporated herein by reference. Additionally, an example of a commercially available product may be the Adaptix™ Interbody System sold by Medtronic Spine and comprising a titanium cage made with Titan nanoLOCK™.

The disclosed implant systems may be employed, for example, with a minimally invasive procedure, including percutaneous techniques, mini-open and open surgical techniques to deliver and introduce instrumentation and/or one or more spinal implants at a surgical site within a body of a patient, for example, a section of a spine. In some embodiments, the implant system may be employed with surgical procedures, as described herein, and/or, for example, corpectomy, discectomy, fusion and/or fixation treatments that employ spinal implants to restore the mechanical support function of vertebrae. In some embodiments, the implant system may be employed with surgical approaches, including but not limited to: anterior lumbar interbody fusion (ALIF), direct lateral interbody fusion (DLIF), oblique lateral lumbar interbody fusion (OLLIF), oblique lateral interbody fusion (OLIF), transforaminal lumbar Interbody fusion (TLIF), posterior lumbar Interbody fusion (PLIF), various types of posterior or anterior fusion procedures, and any fusion procedure in any portion of the spinal column (sacral, lumbar, thoracic, and cervical).

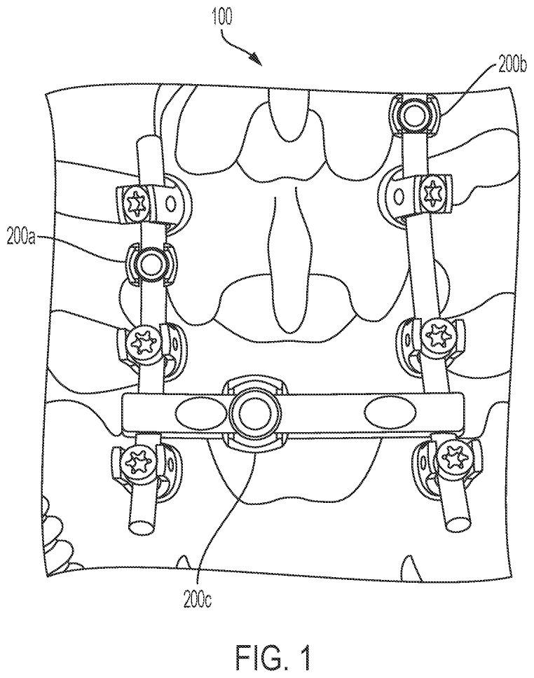

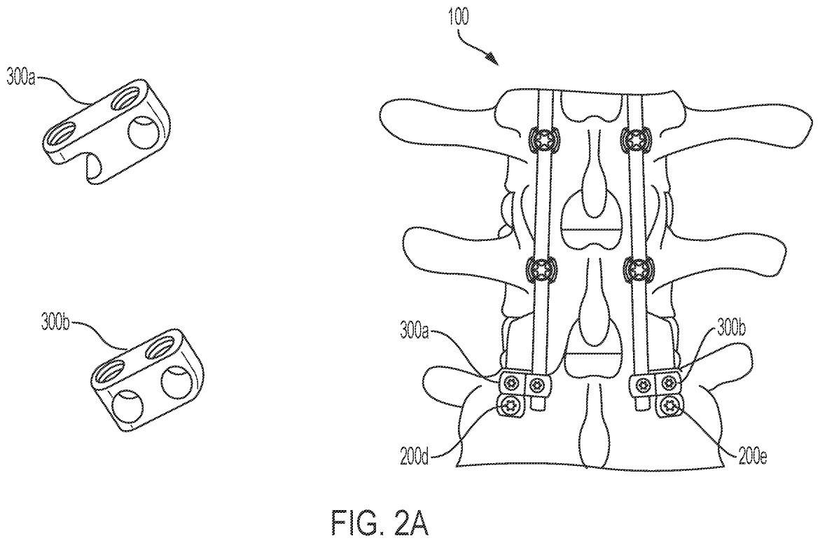

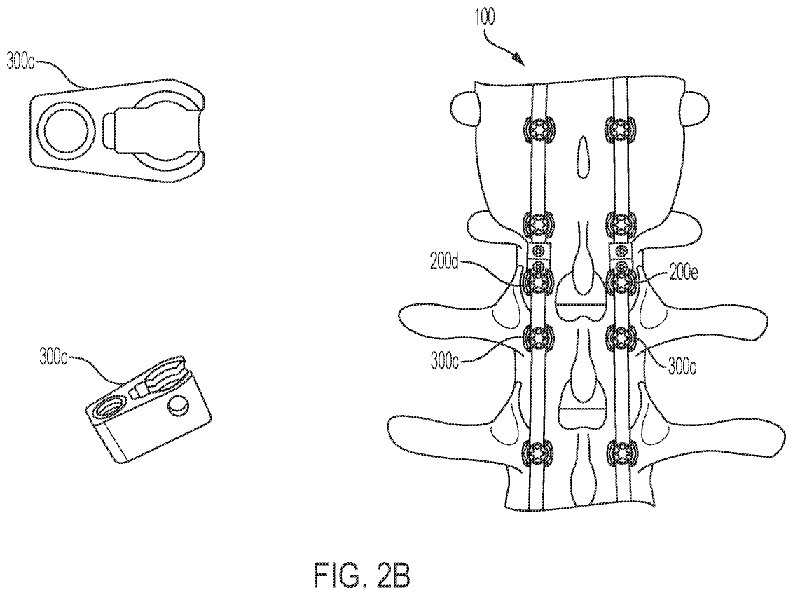

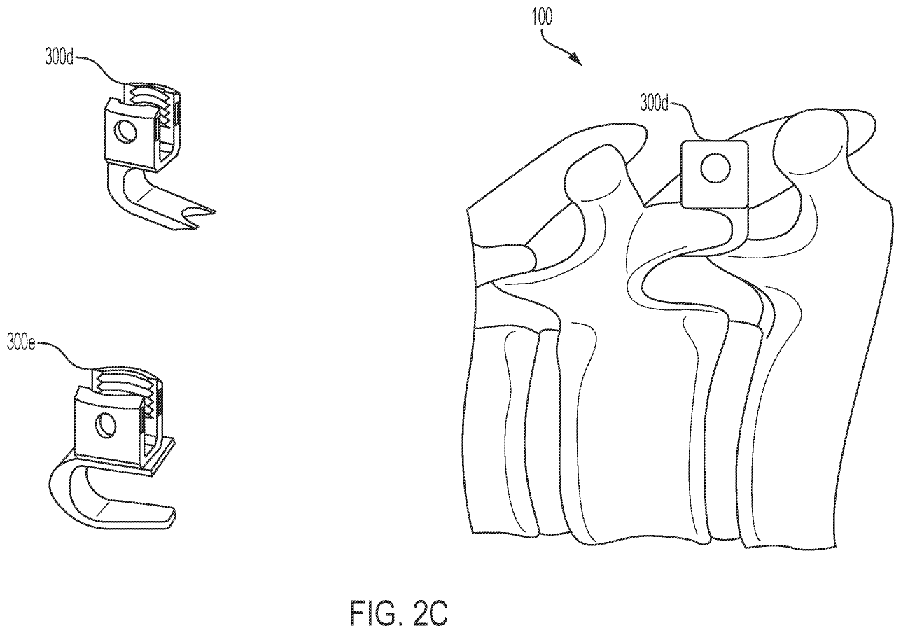

FIG. 1 shows an example surgical construct 100 including alternative placement options for one or more sensor assemblies 200, 200a-c. The surgical construct 100 may include components 300 such as pedicle screws, iliac screws or other bone screws, transverse process hooks, rods, cross connectors, accessories, and so forth. Example components 300, 300a-e are depicted in FIGS. 2A-2C. In general, spinal constructs 100 provide spinal stability/support, limit undesirable movement, redistribute forces among vertebrae, and/or apply corrective forces to vertebrae. Construct design requires understanding of the condition being treated and the biomechanical forces acting on the spine (and on the installed construct 100). Sensor assemblies 200 may provide valuable post-operative information (e.g., in the form of telemetry) regarding the status of the construct 100 and/or the surgical procedures (e.g., spinal fusion). Sensor assemblies 200 that are configured for use in alternative locations may provide otherwise unavailable status information. As shown, these alternative placement options are not limited to typical bone screw shank location. Instead, the sensor assemblies 200a-c are configured to attach to other portions of the construct 100. For example, sensor assembly 200a is attached to a spinal rod, but is not attached to a vertebra. That is, the primary role of sensor assembly 200a is providing telemetry, rather than fixation. As shown, sensor assembly 200a only includes one attachment means, so that it may be attached to the spinal rod. Therefore, sensor assembly 200a has a more compact form than a dual purpose fixation/telemetry assembly. As such, sensor assembly 200a may be positioned in confined spaces, e.g., closer to the patient's spine to provide telemetry information that would not otherwise be available. Thus, sensor assembly 200a provides greater placement flexibility with respect to anatomical constraints. Sensor assembly 200b is attached (axially) to the end of a spinal rod. Similar to sensor assembly 200a, sensor assembly 200b may also be positioned in confined spaces or areas of particular interest and/or provide alternative physiological sensing information from the alternative placement. Sensor assembly 200c is attached to a cross-link connect, an alternative stabilization member which connects two longitudinal rods. Thus, sensor assembly 200c may also provide alternative physiological sensing information. Other alternative placement option include attaching sensor assemblies 200 to auxiliary (e.g., temporary or provisional) longitudinal rods, lateral pelvic connectors, “domino” connectors (which extend perpendicularly away from longitudinal rods), and so forth. These and other alternative locations may allow for telemetry of sensed data that is unavailable from typical locations, e.g., associated with affixing implants to vertebrae.

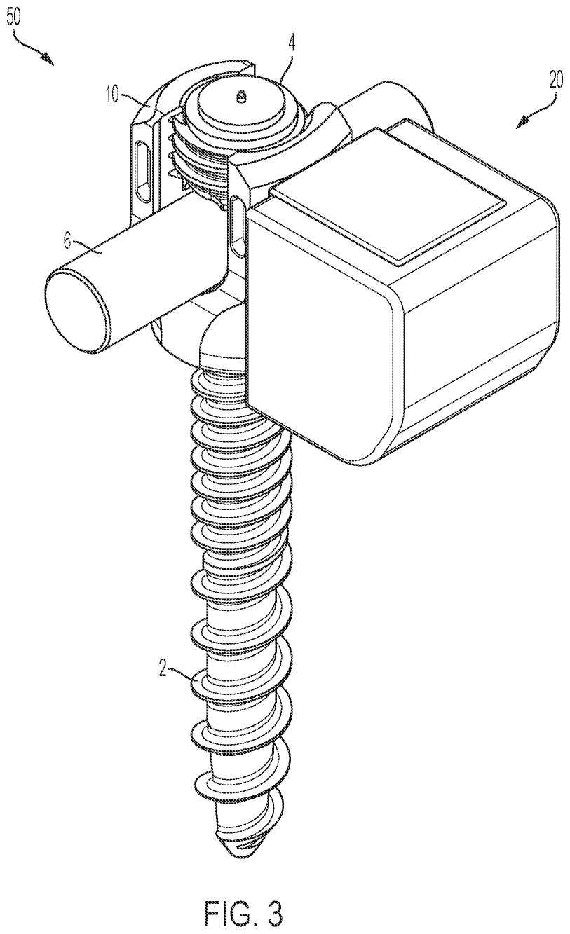

FIG. 2A shows example “domino” connectors 300a, 300b. Domino connectors 300a, 300b may be configured to connect, e.g., adjacent spinal fusion rods having similar or differing diameters. The domino connectors 300a, 300b may be configured to capture one or more longitudinal rods within a circular or semicircular opening. In some examples, a screw (e.g., a set screw) secures the longitudinal rod within the opening. In the alternative placement approach illustrated in FIG. 1, domino connectors 300a, 300b may be configured to connect a spinal rod (or other implant component 300) to a sensor assembly (e.g., 200d, 200e). In some examples, domino connectors 300a, 300b are configured to connect, e.g., the spinal rod, to a short stub rod, i.e., a longitudinal rod just long enough for a sensor assembly (e.g., 200d, 200e) to connect to. The sensor assemblies (e.g., 200d, 200e) may be configured to connect to the stub rod in a similar fashion to how the sensor assemblies (e.g., 200d, 200e) are configured to connect to the longitudinal rod. That is, the sensor assemblies (e.g., 200d, 200e) may include a circular or semi-circular opening to secure the sensor assemblies (e.g., 200d, 200e) to the stub rod. In other implementations, the sensor assemblies (e.g., 200d, 200e) include a U-shaped channel to secure the longitudinal rod, similar to the pedicle screw system 50 of FIG. 3.

The stub rod may serve only as an alternative mounting point for the sensor assembly 200 and not, e.g., to transmit forces from the construct to the patient's anatomy. In some examples, sensor assemblies (e.g., 200d, 200e) connect directly to domino connectors 300a, 300b without a stub rod (or may be integrated with the domino connectors 300a, 300b, e.g., to form a monolithic component). In these and other cases, the sensor assembly (e.g., 200d, 200e) is located adjacent to the longitudinal rod. The sensor assembly (e.g., 200d, 200e) may be located at any useful position with respect to the longitudinal rod, including above, below, or to the side of the longitudinal rod.

Similar to domino connectors 300a, 300b, cross links may be configured to connect parallel longitudinal rods. The longitudinal rods may be spaced somewhat further apart, rather than substantially adjacent. In some examples, sensor assemblies (e.g., 200c, FIG. 1) may connect to the cross link by capturing the cross link within an opening or cavity, such as the circular, semi-circular, U-shaped, or other opening configuration disclosed above, and may secure the cross link in the opening using a screw (e.g., set screw), as disclosed above. Sensor assemblies (e.g., 200c) may also be integrated with cross links, e.g., to form a monolithic component.

FIG. 2B shows example axial connectors 300c. Similar to domino connectors 300a, 300b, axial connectors 300c may also be configured to capture one or more longitudinal rods within a circular or semicircular opening, and may be configured to connect rods having similar or differing diameters. In some examples, a screw (e.g., a set screw) secures the longitudinal rod within the opening. However, while domino connectors 300a, 300b generally connect adjacent longitudinal rods, axial connectors 300c generally connect longitudinal rods having the same or similar longitudinal axis. In some examples, axial connectors 300c are configured to connect, e.g., the spinal rod, to a short stub rod, i.e., a longitudinal rod just long enough for a sensor assembly (e.g., 200d, 200e) to connect to, the stub rod extending away from the spinal rod substantially along the longitudinal axis. In some examples, sensor assemblies (e.g., 200d, 200e) connect directly to axial connectors 300c without a stub rod (or may be integrated with the axial connectors 300c, e.g., to form a monolithic component). In these and other cases, the sensor assembly (e.g., 200d, 200e) is located beyond the end of the longitudinal rod and along the longitudinal axis of the longitudinal rod. The sensor assembly (e.g., 200d, 200e) may connect to the stub rod or to the longitudinal rod in any of the ways disclosed above.

FIG. 2C shows example hooks 300d, 300e. Hooks 300d, 300e may be configured to attach to bones without the use of a bone screw. In some examples, hooks 300d, 300e are configured to connect to a longitudinal rod. Similar to the other components disclosed above, sensor assemblies 200 may connect to the hooks 300d, 300e via a stub rod or directly to hooks 300d, 300e without a stub rod. Sensor assemblies 200 may also be integrated with hooks 300d, 300e, e.g., to form a monolithic component.

FIG. 3 illustrates an example digital pedicle screw system 50 with active sensing ability. The example system 50 is configured to perform fixation as well as telemetry. That is, the system may be attached to a vertebra and to a spinal construct. For example, the digital pedicle screw system 50 may be secured to a vertebra using the pedicle screw 2, and to a longitudinal rod 6 using the receiver 10. As illustrated in FIG. 3, system 50 may include a pedicle screw 2 and a receiver 10 having a side portion 20 for supporting various electronic components and sensors as will be explained in further detail below. The pedicle screw 2 may have a thread pitch extending along a length thereof for implanting and securing the pedicle screw 2 into patient anatomy, e.g., a vertebral body. The pedicle screw 2 may include a head portion (not shown) that may couple to the receiver 10 in a lower cavity (not shown). In various embodiments, a lower cavity of receiver 10 may include at least one annular groove for supporting a deformable annular ring or c-ring that captures the head of pedicle screw 2. In this way, receiver 10 may be popped on to the head of a pedicle screw 2 simply by pressing down on receiver 10 as would be understood by a person of ordinary skill in the art.

In various embodiments, the lower cavity and head may be configured to enable coupling of receiver 10 in a multitude of angled orientations with respect to the extension direction of pedicle screw 2. For example, receiver 10 may be configured as a multiaxial receiver. In other embodiments, receiver 10 may be configured as a monoaxial receiver. In various embodiments a saddle may be disposed within the lower cavity of receiver 10 to support a longitudinal rod 6 disposed in the U-shaped cavity of receiver 10. A set screw 4 may engage to threads of each respective arm of the U-shaped cavity of receiver 10. When sufficiently tightened, set screw 4 may immobilize and/or secure the longitudinal rod 6 within the U-shaped cavity of receiver 10.

Receiver 10 may be coupled to side portion 20, e.g., via a beam portion. In various embodiments, receiver 10 and side portion 20 may be monolithically formed as a single piece or receiver 10 and side portion 20 may be separable pieces that are connected together. In the example embodiment, receiver 10 and side portion 20 are monolithically formed and/or integrally formed together. For example, the receiver 10 is integrally formed with the side portion 20 and they are connected via the beam portion. This arrangement may have the advantage of facilitating the transfer of stress and strain between the receiver 10 and side portion 20 as will be explained in further detail below.

The digital pedicle screw system 50 shown in FIG. 3 performs two roles: fixation and telemetry. Therefore, it includes elements that are unnecessary for a sensor system whose primary purpose is telemetry, rather than fixation. For example, two separate attachment means (pedicle screw 2 and receiver 10) may be unnecessary for a telemetry-oriented sensor system. Furthermore, the relatively large size of the pedicle screw system 50 may render it more awkward to manipulate and/or preclude its use in confined areas, such as the space between a spinal construct and a part of the patient's anatomy, e.g., the patient's spine. An improved sensor system excludes elements that are unnecessary or impede its primary purpose of providing telemetry.

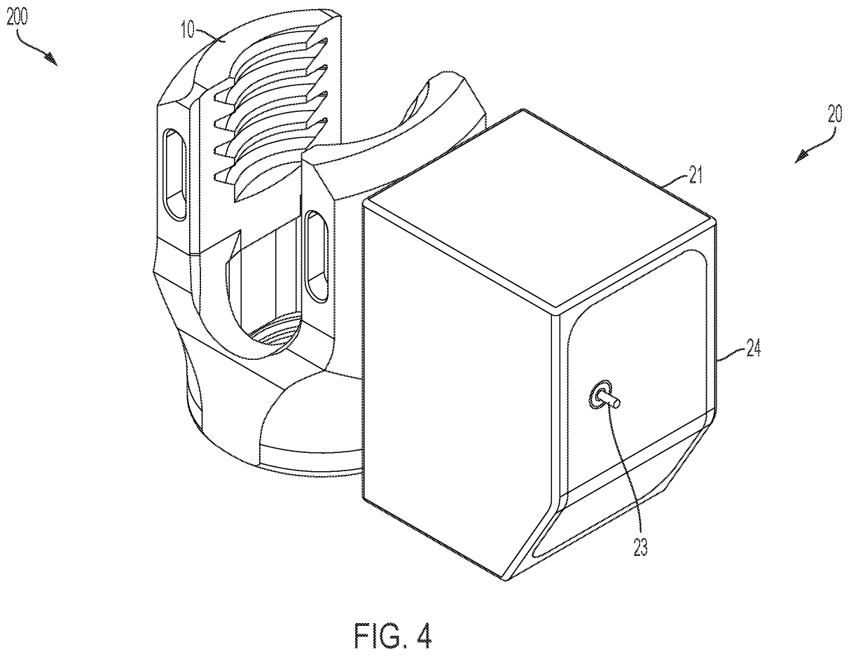

FIG. 4 illustrates an example sensor assembly 200 configured for attachment to a longitudinal rod 6 (or similar structure) and to provide telemetry, but not for fixation. Side portion 20 includes a housing 21 that forms a hermetically sealed cavity therein for housing various microelectronics and sensors. Some example sensors include a strain sensor (also referred to as a stress gauge), accelerometer, gyroscope, temperature gauge, and impedance sensor.

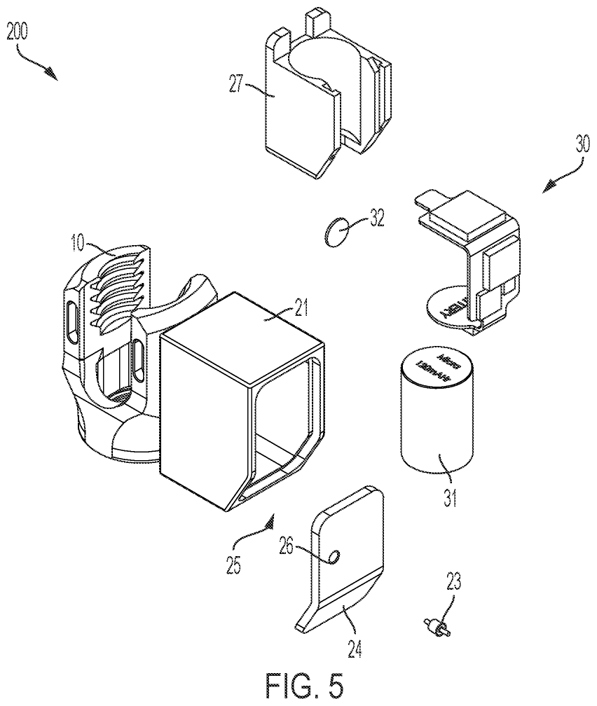

In the example embodiment, a microelectronics assembly 30 (FIG. 5) and battery 31 (FIG. 5) may be disposed inside of a cavity 25 (FIG. 5) and the cavity 25 may be sealed off by cover 24. Cover 24 may have a size and shape corresponding to an opening in housing 21 that exposes the cavity 25 therein. Due to the hermetically sealed nature of cavity 25, a feed-through connection 23 having suitable waterproof flanges may extend through an aperture 26 (FIG. 5) of cover 24. In this way, the feed-through connection 23 may be electrically connected to the microelectronics assembly 30 and an external antenna portion 22 (not shown) while ensuring that a hermetic seal of the electronics components is possible.

FIG. 5 shows an exploded view of an example sensor assembly 200. Housing 21 may define a cavity 25 therein for supporting various electronic components assembled in a microelectronics assembly 30 and a battery 31. In various embodiments, cavity 25 of housing 21 may be hermetically sealed such that the microelectronics assembly 30 and battery therein will not harm a patient when the sensor assembly 200 is installed within the human body. The battery 31 and microelectronics assembly 30 may be installed within the cavity 25 in any suitable way. In the example embodiment, frame 27 may support the battery 31 and microelectronics assembly 30 securely within the cavity such that the microelectronics, battery 31, sensor 32 (e.g., strain gauge, temperature sensor), and antenna portion 22 are electrically connected.

The external antenna portion 22 may include a monopole or “whip” antenna made of a flexible material and is configured to be free floating. That is, the antenna may extend away from the sensor assembly 200 and into the surrounding tissue and be free to move with respect to the surrounding tissue. The tissue surrounding the sensor assembly 200, consisting primarily of muscle tissue, may tolerate a flexible antenna more than more sensitive tissue (e.g., nerves and/or blood vessels). The antenna may also extend toward the patient's skin, i.e., closer to an external reader. By extending closer to an external reader, the transmitted signal will have less tissue to pass through to reach the reader and, therefore, the signal will be less attenuated than a similar signal transmitted from a corresponding internal antenna of the sensor assembly 200.

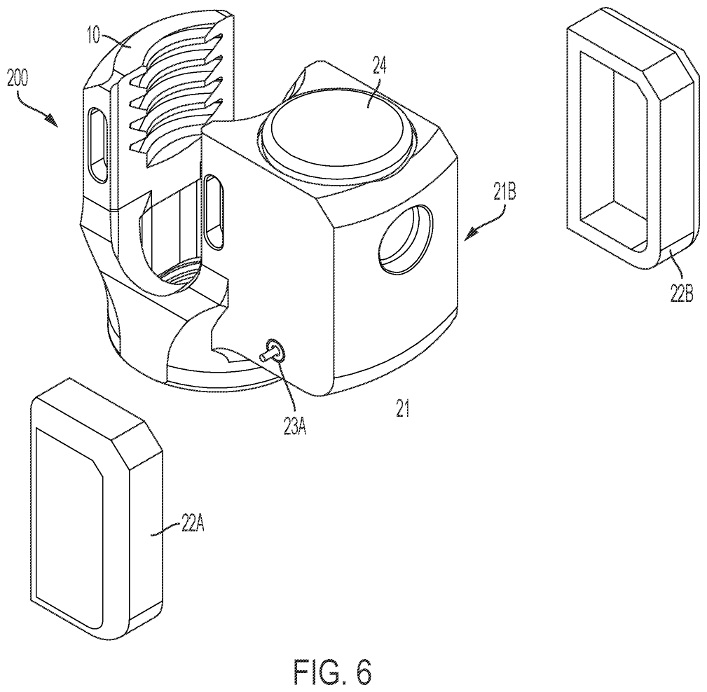

In some embodiments, the antenna portion 22 may be included within or closely adjacent to the cavity. For example, FIG. 6 shows an exploded view of an example sensor assembly 200. In the example embodiment, the microelectronics assembly 30 and battery 31 may be disposed inside of the cavity 25 and the cavity 25 may be sealed off by cover 24. In this embodiment, cover 24 is a circular plate that is dimensioned to cover a corresponding opening in the top portion of housing 21. Additionally, in this embodiment, a first antenna portion 22A is disposed on a first sidewall 21A of housing 21 and a second antenna portion 22B is disposed on a second sidewall 21B of housing 21. For example, the first antenna portion 22A is disposed on an opposite sidewall of the housing 21 opposite the second antenna portion 22B. In this embodiment, the antenna portions 22A and 22B include a corresponding cavity for housing any suitable type of antenna, e.g., a grid antenna or a patch antenna and/or any combination of antennas as explained previously. In some embodiments, each cavity of the antenna portions 22A, 22B may house different types of antenna having different communication frequencies and protocols.

A first feed-through connection 23A having suitable waterproof flanges may extend through first sidewall 21A and a second feed-through connection 23B (not shown) having suitable waterproof flanges may extend through second sidewall 21B. In this way, the feed-through connections 23A and 23B may be electrically connected to the microelectronics assembly 30 and the antenna portions 22A, 22B while ensuring that a hermetic seal of the electronics components is possible.

Various antenna and communication types may be, for example, MICS and BLE. As used herein, “MICS” may refer to the Medical Implant Communication System which may be a short-range communication technology that operates at a frequency from about 402 to 405 MHz. As used herein, “BLE” may refer to Bluetooth low energy communication standard. In some embodiments, the antenna may be a multi-band electrically coupled loop antenna (ECLA) antenna capable of operating in at least the MICS and LBE bands.

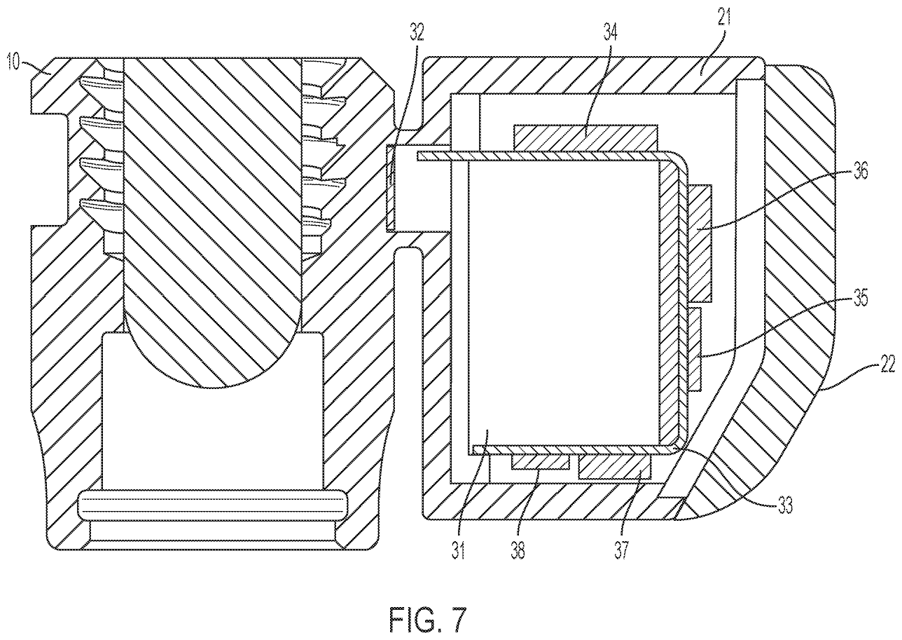

As shown in FIG. 7, the microelectronics assembly 30 may have great variability in the types of circuitry and hardware. Example electronics components may include a flexible circuit board 33 providing an electrical connection between the battery 31, strain gauge 32, and the various other electronics components. A non-limiting list of example electronics components may include an Application Specific Integrated Controller (ASIC) 34, micro controller 35, a wake-up sensor 36, a memory storage 37, and a temperature sensor 38. Example electronics components may include a (flexible) circuit board providing an electrical connection between the battery 31, sensor 32 (e.g., strain gauge, temperature sensor), and the various other electronics components. A non-limiting list of example electronics components may include a mainboard or other suitable printed circuit board (PCB), an application specific integrated circuit (ASIC), a micro controller, a wake-up sensor, a memory storage, a charge storage capacitor, and various mechanical electrical sensors or micro electromechanical systems (MEMs). Example MEMs may include a strain gauge, an impedance sensor, and/or a temperature sensor. However, other MEMs sensors may be incorporated in other embodiments depending on the particular use case.

In various embodiments the memory storage may be a non-transitory memory data store that may store information and/or data from various sensors and electronics components. For example, one or more measurements of a sensor 32 (e.g., strain gauge, temperature sensor) may be stored in memory storage. As another example, a unique identifier associated with a load sensing assembly, a component thereof, or a set screw 4 may be stored in memory. One or more measurements received from sensor 32 may be used to make determinations of the condition of the sensor assembly 200 and/or treatment of a spinal disorder. For instance, proper placement of a longitudinal member 6, set screw 4 and/or pedicle screw 2 may result in an acceptable range of force measurements collected by a strain gauge. Measurements outside of this range may indicate a problem with the placement or positioning of the longitudinal member 6, set screw 4 and/or pedicle screw 2. For example, loosening of a critical component, construct failure, yield or fracture/breakage, improper torque, breakage of the bone segment or portion, the occurrence of fusion or amount of fusion, and/or the like.

In various embodiments, one or more measurements obtained by sensors 32 (e.g., strain gauge, temperature sensor) may be stored by an integrated circuit of a corresponding load sensing assembly such as, for example, in non-transitory computer readable memory storage disclosed above. In this way, the sensor assembly 200 may be continuously powered by the battery 31 and obtain measurements over time. In some embodiments, the sensor assembly 200 may “wake-up” at predetermined time periods to record various data points at predetermined time intervals. For example, the sensor assembly 200 may be programmed to wake up at one-hour intervals, two hour intervals, etc. and record various data points to the memory storage. In this way, the power of the battery 31 may be preserved.

In various embodiments, an antenna and/or wake up sensor may be interrogated by an external reader device (not illustrated) which may cause the transmission of data stored in the memory storage. In this embodiment, the sensor assembly 200 may not continuously transmit data stored in the memory storage, but rather may only transmit data stored in the memory storage when interrogated by a reader. For example, transmission of data may occur in response to being interrogated by the reader, or the transmission may be initiated at timed intervals. In various embodiments, the reader may receive the transmitted measurements, which may be displayed to a user such as a physician. Example readers may include at least one antenna for receiving and/or transmitting data across a suitable bandwidth and protocol similar to or the same as antenna. A reader may also include a central processing unit CPU, and a non-transitory computer readable medium (such as a memory unit or memory cell storing programmable computer implemented instructions).

In various embodiments, one or more measurements obtained by strain gauge 32 may be stored by an integrated circuit of a corresponding load sensing assembly such as, for example, in non-transitory computer readable memory as disclosed above. In turn, antenna and/or electronics components 30 may be interrogated by a reader. For instance, an RFID chip may be read by an RFID reader. As another example, an NFC chip may be read by or may otherwise communicate with an NFC reader or other NFC-enabled device. In other embodiments, a custom protocol may be used, for example a 125 kHz inductive link. Example readers may include at least one antenna for receiving and/or transmitting data with antenna, a central processing unit CPU, and a non-transitory computer readable medium (such as a memory unit or memory cell storing programmable computer implemented instructions). In at least one embodiment, an electromagnetic reader (first reader) may transmit electromagnetic energy to the sensor assembly 200 (or pedicle screw system 50) to power electronic components 30. An RFID reader or an NFC reader may be used separately to read, acquire, and/or interpret data received from antenna. A reader may interrogate an integrated circuit when in a certain proximity to the integrated circuit. In certain embodiments, a reader may interrogate an integrated circuit that has been implanted into a patient as part of a sensor assembly 200 (or pedicle screw system 50), causing the integrated circuit to transmit one or more measurements to the reader. In other embodiments, an integrated circuit may communicate with a reader or other electronic device without being interrogated. The reader may receive the transmitted measurements, and may cause at least a portion of the measurements to be displayed to a user. For instance, a physician may use a reader to interrogate an RFID chip of a patient's implant. The reader may include a display, or may be in communication with a display device, which may display at least a portion of the measurements received from the RFID chip.

In various embodiments, one or more sensors of electronic components 30 may transmit information by directly modulating a reflected signal, such as an RF signal. The strain gauge (or other) sensors 32 may form a Wireless Passive Sensor Network (WPSN), which may utilize modulated backscattering (MB) as a communication technique. External power sources, such as, for example, an RF reader or other reader, may supply a WPSN with energy. The sensor(s) of the WPSN may transmit data by modulating the incident signal from a power source by switching its antenna impedance.

In another embodiment, an integrated circuit may be active, meaning that the chip is battery-powered and capable of broadcasting its own signal. An active integrated circuit may transmit information in response to be interrogated by a reader, but also on its own without being interrogated. For instance, an active integrated circuit may broadcast a signal that contains certain information such as, for example, one or more measurements gathered by an associated strain gauge. An active integrated circuit may continuously broadcast a signal, or it may periodically broadcast a signal. Power may come from any number of sources, including, for example, thin film batteries with or without encapsulation or piezo electronics.

One or more measurements received from a load sensing assembly may be used to make determinations of the condition of a spinal implant and/or treatment of a spinal disorder. For instance, proper placement of a longitudinal member 6, set screw 4 and/or anchoring member may result in an acceptable range of force measurements collected by a strain gauge of a load sensing assembly. Measurements outside of this range may indicate a problem with the placement or positioning of a longitudinal member, set screw and/or anchoring member such as, for example, loosening of a set screw 4 and/or anchoring member, longitudinal member failure, construct failure, yield or fracture/breakage, improper torque, breakage of the bone segment or portion, the occurrence of fusion or amount of fusion, and/or the like. In these instances, the reader may contain a range of pre-determined acceptable values corresponding to the strain gauge and/or other MEMs sensors 32. If the actual measured reading of the strain gauge and/or other MEMs sensors 32 falls outside of the range, the reader may notify an end user, a hospital management system, and/or the patient. For example, a patient may continuously or regularly monitor the actual measured readings of sensor assembly 200 (or pedicle screw system 50) on an outpatient basis with a reader. In some embodiments, a reader may be configured to relay information received from antenna to a secondary processing component such as an external display, computer, server, hospital management system, or other type of data processing equipment. The secondary processing component may process information received by the reader from antenna via a processor, controller, and memory configured to execute programmable computer implemented instructions. In this way, disclosed systems increase the likelihood that a patient can detect a malfunction, such as loosening of a set screw 4, before catastrophic failure.

One or more tools or instruments may include a reader which may be used to gather information from one or more integrated circuits of electronic components 30 during or in connection with a procedure. For instance, a torque tool (not illustrated) may be used to loosen or tighten the set screw 4. A torque tool may include a reader, or may be in communication with a reader, such that a user of the torque tool is able to obtain, in substantially real time, one or more measurements relating to the set screw 4 and longitudinal rod 6 placement that are measured by a strain gauge 32 of a load sensing assembly of the set screw 4 via the tool. For instance, as a user is applying torque to a set screw 4, the user may see one or more force measurements between the set screw 4 and the longitudinal member 6 in order to determine that the positioning of the set screw 4 and/or longitudinal member 6 is correct and that the proper force is being maintained. In certain embodiments, a tool or instrument may include a display device (not illustrated) on which one or more measurements may be displayed. In other embodiments, a tool or instrument may be in communication with a display device (not illustrated), and may transmit one or more measurements for display on the display device via a communications network.

In some embodiments, an electronic device, such as a reader or an electronic device in communication with a reader (not illustrated), may compare one or more measurements obtained from an integrated circuit to one or more acceptable value ranges. If one or more of the measurements are outside of an applicable value range, the electronic device may cause a notification to be made. For instance, an electronic device may generate an alert for a user, and cause the alert to be displayed to the user via a display device. Additionally or alternatively, an electronic device may send an alert to a user such as via an email message, a text message, a notification, or otherwise.

An integrated circuit of electronics components 30 may store a unique identifier associated with the implant components 300 to which the load sensing assembly corresponds. For example, an integrated circuit of electronics components 30 for a sensor assembly 200 (or pedicle screw system 50) may store a unique identifier associated with the sensor assembly 200 (or pedicle screw system 50). For example, when a reader interrogates an integrated circuit, the integrated circuit may transmit a unique identifier for an implant component 300 that is stored by the integrated circuit to the reader. Having access to a unique identifier for an implant component 300 may help a user ascertain whether the measurements that are being obtained are associated with the implant component 300 of interest. Also, having access to a unique identifier for an implant component 300 may help a user take inventory of one or more implant components 300. For instance, after spinal surgery, a physician or other health care professional may use a reader to confirm that all of the implant components 300 allocated for the procedure have been used and are positioned in a patient. This may also help with the detection and verification of screws in a patient's body.

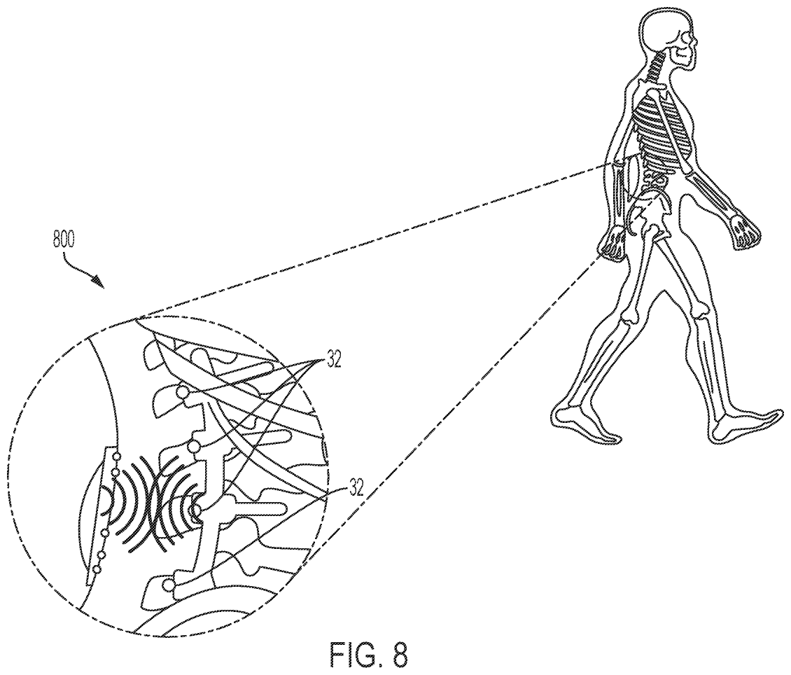

FIG. 8 illustrates an example of a surgical site (SS) monitoring system 800 that may utilize example digital pedicle screw systems 50 and/or sensor assembly 200 disclosed herein. In some embodiments, the SS monitoring system 800 may be a surgical site load monitoring system (using one or more strain gauges 32) and/or an infection monitoring system (using one or more temperature sensors 32). In at least one embodiment, a temperature sensor is positioned to discern a body temperature of a patient in the region of the sensor assembly 200, and in others a temperature sensor is positioned to discern a body temperature of a patient in a region adjacent a portion of the assembly 200 that is directly exposed to patient tissue or contacting a portion of a longitudinal rod 6. FIG. 8 illustrates a single implant system (e.g., a single spinal-fusion construct 100) having multiple separate sensor-equipped implants, each of which may have one or more sensors. Other embodiments within the scope of this disclosure include a single sensor-equipped implant having one or more sensors 32. Still other embodiments include multiple implant systems, e.g., multiple spinal-fusion constructs, or one spinal-fusion construct 100 and a separate sensor-equipped implant. Other combinations and permutations of implant systems and/or sensor-equipped implants are also within the scope of the disclosure.

In one or more embodiments, the SS monitoring system 800 may include an array of implants, in which one or more of the implants have any type of MEMs sensor 32 as previously disclosed. For the cases in which the SS monitoring system 800 includes an array of implants having various MEMs sensors 32, the received data from the one or more MEMs sensors 32 may be compared to one another to diagnose the quality of the surgical procedure, the integrity of the implant, and/or an infection at the surgical site.

It should be understood that various aspects disclosed herein may be combined in different combinations than the combinations specifically presented in the description and accompanying drawings. For example, features, functionality, and components from one embodiment may be combined with another embodiment and vice versa unless the context clearly indicates otherwise. Similarly, features, functionality, and components may be omitted unless the context clearly indicates otherwise. It should also be understood that, depending on the example, certain acts or events of any of the processes or methods described herein may be performed in a different sequence, may be added, merged, or left out altogether (e.g., all described acts or events may not be necessary to carry out the techniques).

The breadth and scope of this disclosure should not be limited by any of the above-described example embodiments, but should be defined only in accordance with the following claims and their equivalents.

What is claimed is:

1. A spinal implant, comprising:
an attachment portion having an opening configured to capture a longitudinal member therein, wherein the attachment portion is the only attachment means of the spinal implant;
a housing integrally connected to the attachment portion and defining a sealed cavity for supporting a microelectronics assembly and a power source therein;
at least one antenna in electrical communication with the microelectronics assembly; and
at least one sensor in electrical communication with the microelectronics assembly, wherein the microelectronics assembly is configured to transmit information received from the at least one sensor to an external device using the at least one antenna.

2. The spinal implant of claim 1, wherein the spinal implant is configured to extend away from the longitudinal member in an axial direction when attached to the longitudinal member.

3. The spinal implant of claim 1, wherein the attachment portion comprises a hook.

4. The spinal implant of claim 1, wherein the at least one antenna is disposed within the housing.

5. The spinal implant of claim 1, wherein the at least one antenna comprises a first antenna portion and a second antenna portion disposed on opposite sides of the housing.

6. The spinal implant of claim 1, wherein the power source is a battery.

7. The spinal implant of claim 6, wherein the battery is configured to be inductively recharged.

8. The spinal implant of claim 1, wherein the housing comprises a hermetically sealed, removable electronics cartridge having at least one antenna interface.

9. The spinal implant of claim 8, wherein the removable electronics cartridge has a threaded exterior that mates with the housing.

10. The spinal implant of claim 8, wherein the at least one sensor is disposed within the removable electronics cartridge.

11. The spinal implant of claim 1, wherein the at least one antenna is configured to flexibly extend away from the spinal implant.

12. The spinal implant of claim 1, wherein the at least one sensor comprises at least one temperature sensor configured to measure a temperature of a surgical site.

13. The spinal implant of claim 1, wherein the at least one sensor comprises at least one strain gauge configured to measure a localized force sensed by the spinal implant.

14. The spinal implant of claim 1, wherein the at least one sensor is chosen from the group comprising: accelerometer, gyroscope, strain gauge, pressure sensor, pH sensor, impedance sensor, optical sensor, and temperature sensor.

15. The spinal implant of claim 1, wherein the opening is a threaded U-shaped cavity.

16. The spinal implant of claim 15, wherein the attachment portion further comprises a threaded portion configured to receive a screw, thereby securing the spinal implant to the longitudinal member.

17. A surgical site monitoring system comprising:
the spinal implant of claim 1; and
an external reader device configured to receive the information transmitted from the spinal implant.

18. The surgical site monitoring system of claim 17, wherein the external reader device is configured to display at least a portion of the information transmitted from the spinal implant.

19. The surgical site monitoring system of claim 17, wherein the external reader device is configured to provide a notification when one or more measurements transmitted from the spinal implant are outside of a value range.

20. The surgical site monitoring system of claim 17, wherein the external reader device is configured to receive the information through an intermediate relay device.
